(19) United States
(12) Patent Application Publication
(10) Pub. No.: US 2007/0197485 A1
(43) Pub. Date: Aug. 23, 2007

(54) Therapy and Use of Compounds in Therapy

(75) Inventors: Stefan Anker, Berlin (DE); Andrew Coats, London (GB); Hans-Dieter Volk, Berlin (DE); Ralf Schuman, Berlin (DE); Mathias Plauth, Berlin (DE); Herbert Lochs, Berlin (DE)

Correspondence Address: NORRIS, MCLAUGHLIN & MARCUS, P.A., 875 THIRD AVE, 18TH FLOOR, NEW YORK, NY, 10022, US

(73) Assignee: MAX-DELBRUECK-CENTRUM FUR MOLEKULARE MEDIZIN, Berlin (DE)

(21) Appl. No.: 11/627,183

(22) Filed: Jan. 25, 2007

Related U.S. Application Data
(63) Continuation of application No. 10/019,452, filed on Feb. 25, 2002, now abandoned.

(30) Foreign Application Priority Data
Mar. 9, 1999 (GB) 9905315.9
Mar. 9, 1999 (GB) 9905300.1
Mar. 9, 1999 (GB) 9905310.0
Mar. 9, 1999 (GB) 9905307.6
Mar. 9, 1999 (GB) 9905314.2

(51) Int. Cl. A61K 31/56

(52) U.S. Cl. 514/169

(57) ABSTRACT

A method for treating cachecia, liver disease, and other wasting syndromes is provided. The treatment method comprises administering a therapeutically effective amount of a compound that is able to reduce the production, absorption and/or the effect of an endotoxin. Examples of such compounds include, but are not limited to, bile acids; bactericidal/permeability increasing proteins; lipoproteins; antibodies capable of binding to endotoxins; LPS binding protein; activated charcoal, Fuller's earth, attapulgite, kaolin or bentonite or a clay; a drug blocking effectively signaling through toll-like receptors; and calostrum or human, bovine or other mammals.

This application is a continuation of U.S. Ser. No. 10/019,452 filed Feb. 25, 2002, which is a 371 application of PCT/EP2000/02062 filed Mar. 9, 2000.

The present invention relates to therapy and the use of agents in the therapy of cachexia and wasting syndromes due to diseases other than congestive heart failure. Cachexia occurs in a number of other chronic diseases, like liver cirrhosis, chronic obstructive pulmonary disease, chronic renal failure, diabetes, rheumatoid arthritis. Cachexia and weight loss are linked to inflammatory processes and they are linked to increased mortality and/or morbidity. Cytokine activation is a potential causal mechanism for the development of cachexia also in these other diseases.

No one has previously proposed that one or all of the following agents may be useful in the management of patients with cachexia due to liver cirrhosis, chronic obstructive pulmonary disease, chronic renal failure, diabetes, rheumatoid arthritis:
a bile acid,
BPI,
LPS binding protein or a functional equivalent thereof,
an antibody capable of binding to endotoxin,
the combination of lipoproteins and LPS binding protein,
activated charcoal, Fuller's earth, attapulgite, kaolin or bentonite or a clay,
an antibody able to bind the CD14 receptor,
a soluble CD14 receptor,
a drug blocking effectively signaling through toll-like receptors, particularly toll-like receptor 4 and 2
colostrum of human, bovine, or other mammalian origin

The following classes of patients in particular may benefit from treatment

1. Patients with liver cirrhosis, chronic obstructive pulmonary disease, chronic renal failure, diabetes, rheumatoid arthritis.

2. Patients with cachexia due to liver cirrhosis, chronic obstructive pulmonary disease, chronic renal failure, diabetes, rheumatoid arthritis.

It is preferred that the patient has cachexia, as characterised by loss of muscle, fat, and or bone tissue.

It is preferred that the patient has experienced weight loss >7.5%.

It is preferred that the compound is able to substantially reduce the biological activity of endotoxin (lipopolysaccharide) such that the endotoxin mediated production of inflammatory cytokines in the circulating blood is reduced.

By “bile acid” we include all naturally occurring bile acids whether from man or from another animal. Also is included bile acids which are synthetic or semi-synthetic derivatives of naturally occurring bile acids. Of course, all bile acids including those that are “naturally occurring” may be synthesised chemically.

Bile acids are available from Falk Pharma. GmbH and are described, for example, in WP96/17859, DE29717252 and WO98/05339.

Bile acids for use in the method of the invention include, but are not limited to, chemodeoxycholic acid (3d,7α-dihydroxy-5-cholan-24-oic-acid), arsodeoxycholic acid (3α,7-dihydroxy-5-cholan-24-oic acid), dehydrocholic acid (3,7,12-trioxo-5-cholan-24-oic acid), cholic acid and deoxycholic acid.

Preferably, the bile acid is a bile acid which is able to form micelles. Preferably, the bile acid is able to form a micelle around an endotoxin (lipopolysacharide molecule). It is particularly preferred that the bile acid is able to bind to endotoxin (lipopolysaccharide) molecules and substantially reduce the available endotoxin in the patient. In particular, it is preferred if the bile acid is able to substantially reduce the biological activity of endotoxin (lipopolysaccharide) such that the endotoxin has a substantially reduced effect on the liver or does not reach the liver in a substantially active form.

It is preferred if the bile acid is any one of ursodeoxycholic acid, chemodeoxycholic acid, dehydrocholic acid, cholic acid and deoxycholic acid.

It is preferred if the bile acid is ursodeoxycholic acid.

Originally, UDCA was registered for the medical treatment of gallstones (Leuschner et al. Our ten year experience in gallstone dissolution. Comparison with the national Canadian gallstone (NCGS, USA) and the Tokyo co-operative gallstone study (TCGS, Japan). Gastroenterology 1982, 82:1113). Ursodeoxycholic acid has for many years been proposed to be useful also in patients with cholestatic disease, and particularly in patients with primary biliary cirrhosis, a chronic cholestatic liver disease (Lindor et al. Effects of ursodeoxycholic acid on survival in patients with primary biliary cirrhosis. Gastroenterology 1996, 110:1515-1518). In analogy, UDCA is used in other cholestatic disorders like primary sclerosing cholangitis (Beuerset al: Therapie der autoimmunen Hepatitis, primar biliaren Zirrhose und primär sklerosierenden Cholangitis. Konsensus der Deutschen Gesellschaft für Verdauungsund Stoffwechsellkrankheiten. Z. Gastroenterologie 1997; 35:1041-1049) or benign cholestasis of pregnancy (Palma et al. Ursodeoxycholic acid in the treatment of cholestasis of pregnancy: a randomized, double-blind study controlled with placebo. J Hepatol 1997, 27:1022-1028). Regarding its mode of action, most authorities regard increased bile flow and a reduced hepatocellular insult as a result of improved bile flow and altered bile salt patterns as the main modes of UDCA action in chronic cholestatic liver diseases.

However, a very recent meta-analysis concluded that “Published randomised controlled trials of UDCA do not show evidence of therapeutic benefit in primary biliary cirrhosis and its use as standard therapy needs to be re-examined.” (Goulis et al. Randomised controlled trials of ursodeoxycholic-acid therapy for primary biliary cirrhosis: a meta-analysis. Lancet Sep. 26, 1999; 354:1053-1060.)

As for other liver diseases another recent review article concluded “Ursodeoxycholic acid is of unproven efficacy in non-cholestatic disorders such as acute rejection after liver transplantation, non-alcoholic steatohepatitis, alcoholic liver disease and chronic viral hepatitis.” Trauner M and Graziadei I W. Review article: mechanisms of action and therapeutic applications of ursodleoxycholic acid in chronic liver diseases. Aliment Pharmacol Ther. August 1999; 13(8): 979-996.

Therefore, treatment with ursodeoxycholic acid (UDCA) can not be considered a treatment with proven efficacy in patients with liver disease.

It has never been suggested that ursodeoxycholic acid (UDCA) should be specifically given to patients with cachexia due to liver cirrhosis.

It has never been suggested that ursodeoxycholic acid (UDCA) should be specifically given to patients with alcoholic liver cirrhosis. In fact, such patients were specifically excluded from studies.

Alterations in nutritional state leading to abnormal body composition are detectable already in early stages of liver cirrhosis and are clinically overt in the great majority of patients with advanced disease. Despite the well accepted prognostic role of cachexia or protein-energy-malnutrition in cirrhosis its pathogenesis is not fully understood. Although alcohol abuse and inadequate nutrient composition may play some role in patients with alcoholic liver disease this clearly is not operative in patients with liver disease of other etiology in whom malnutrition is as great a problem as in those with alcoholic liver disease (Plauth et al: ESPEN guidelines for nutrition in liver disease and transplantation. Clin Nutr 1997, 16:43-55). Nutrient intake is reduced in many patients with advanced liver cirrhosis and does not match requirements. It is unknown, however, whether food intake is reduced as a consequence of mechanical factors such as ascites or due to altered appetite regulation or other processes.

It is long known that endotoxaemia occurs in a number of patients with liver cirrhosis. It is not known, whether endotoxin (LPS) levels are particularly raised in patients with cachexia due to liver cirrhosis.

Depending of the severity of the liver cirrhosis process, cachexia occurs in 30 to 60% of patients with liver cirrhosis, and the survival of patients with cachexia in liver cirrhosis is impaired. (Plauth et al: ESPEN guidelines for nutrition in liver disease and transplantation. Clin Nutr 1997, 16:43-55). There is no known specific therapy for these patients, and randomised placebo controlled clinical trials to reverse the cachexia in liver cirrhosis patients, and particularly in those with alcohol induced liver cirrhosis have not been performed. Additionally, patients with a body cell mass (BCM) <35% of body weight have reduced survival also after liver transplantation, and the 5-year survival rate is 54% compared to 88% in patients with BCM >35% (p<0.01) (Selberg et al, Identification of high- and low-risk patients before liver transplantation: a prospective cohort study of nutritional and metabolic parameters in 150 patients. Hepatology 1997;25:652-657).

It has also been suggested that bile acids can protect the liver against endotoxin action in obstructive jaundice when patients undergo surgery (Greve et al. Bile acids inhibit endotoxin-induced release of tumor necrosis factor by monocytes: an in vitro study. Hepatology Oct. 1, 1989; 10(4):454-458). With regards to monocyte generated cytokine production in response to LPS, in this study deoxycholic acid was the most effective, chenodeoxycholic acid was less effective and ursodeoxycholic acid was ineffective in the concentrations used. Bile acids did not inactivate endotoxin as measured in a chromogenic Limulus amebocyte lysate assay. In these studies patients with non-cholestatic or alcoholic aetiology were not considered, and there was no data or discussion of cachexia and weight loss.

In experiments, rats with obstructive jaundice, LPS was administered via the portal vein. In UDCA-treated rats, the endotoxin concentration was significantly lower, however, that UDCA had no effect on the TNF-alpha levels (Hori Y & Ohyanagi H. Protective effect of the intravenous administration of ursodeoxycholic acid against endotoxaemia in rats with obstructive jaundice, Surg-Today 1997;27:140-144). In a case control study UDCA showed also no clinical benefit in patients with chronic hepatitis C, and serum TNT and IL-6 levels could not be shown to be affected by UDCA treatment (Lu et al. Efficacy of ursodeoxycholic acid in the treatment of patients with chronic hepatitis C. J Gastroenterol Hepatol 1995;10:432-437.

In summary, the immunological effects of ursodeoxycholic acid (UDCA) on plasma LPS and cytokine levels are poor in these studies, and the cellular effects of ursodeoxycholic acid (UDCA) are conflicting.

It is important to note that it has never been proposed that ursodeoxycholic acid (UDCA) should be given in patients with weight loss, i.e. cachexia, in patients with liver disease. It has never been proposed that ursodeoxycholic acid (UDCA) could prevent or reverse weight loss, i.e. cachexia, in patients with liver disease. Additionally, it has never been proposed that ursodeoxycholic acid (UDCA) could prevent or reverse weight loss, i.e. cachexia, in patients with chronic obstructive pulmonary disease, chronic renal failure, diabetes, rheumatoid arthritis.

The invention will now be described by reference to the following additional examples and figures.

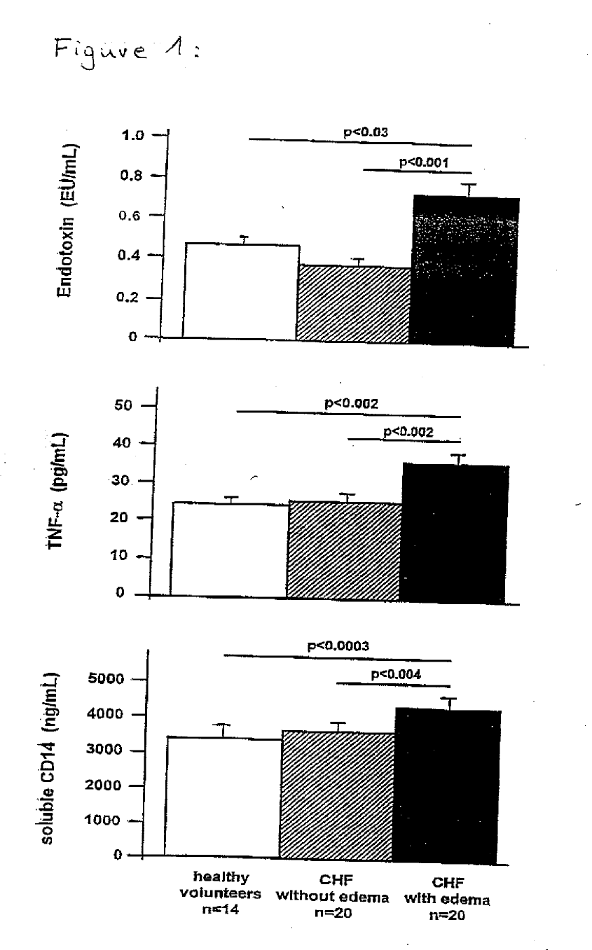

FIG. 1 is a graphical representation showing levels of soluble CD14, TNF-α and endotoxin in a sampling of patients;

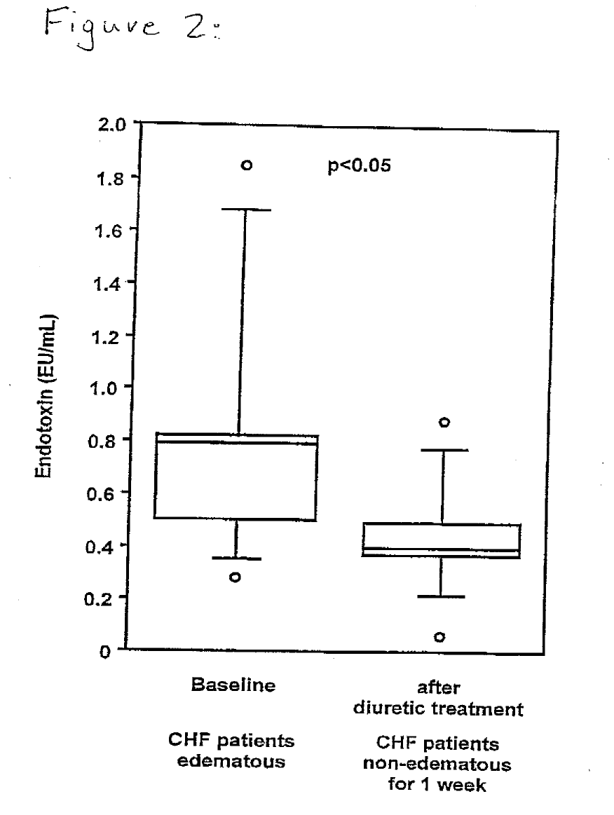

FIG. 2 is graphical representation showing the concentration of endotoxin in CHF patients before and after treatment;

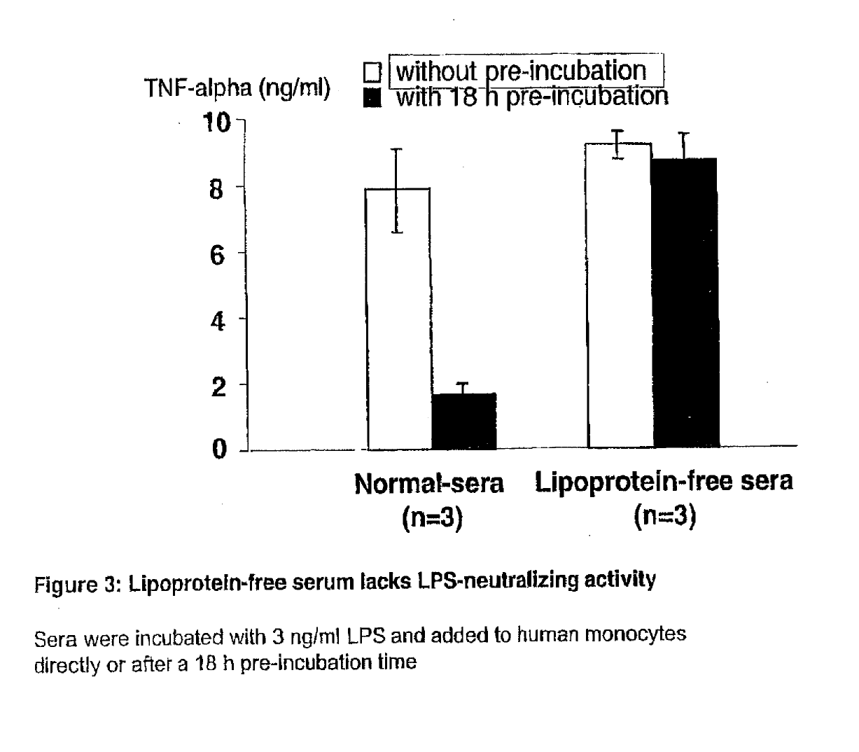

FIG. 3 is a graphical representation showing that Lipoprotein-free serum lacks LPS-neutralizing activity;

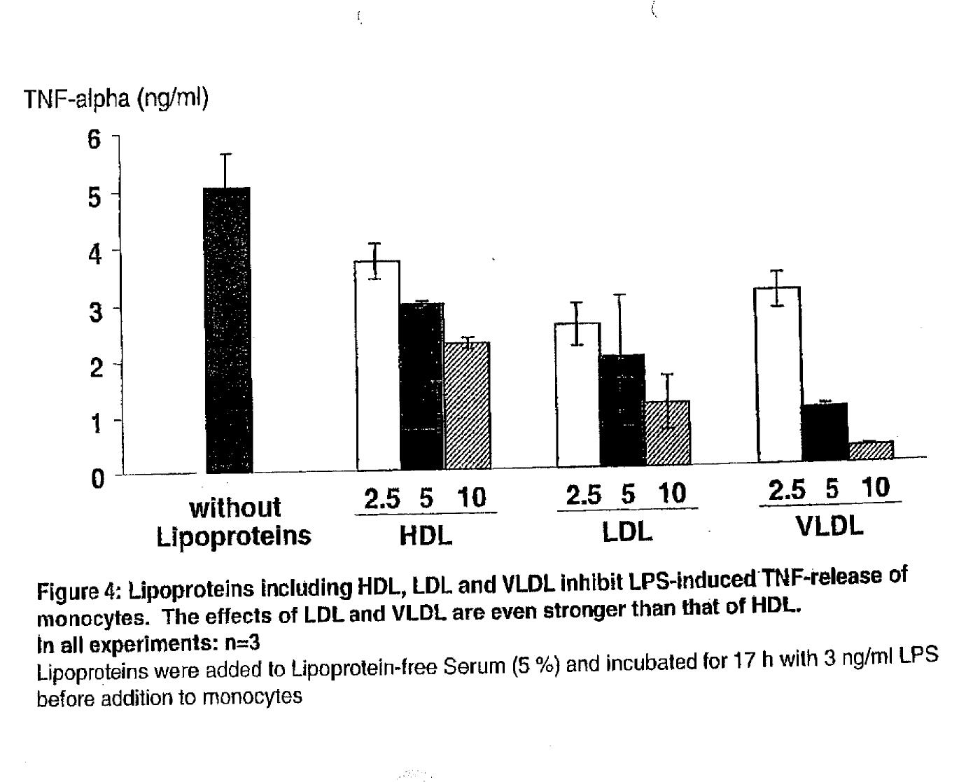

FIG. 4 is a graphical representation showing that certain lipoproteins inhibit LPS-induced TNF-α release;

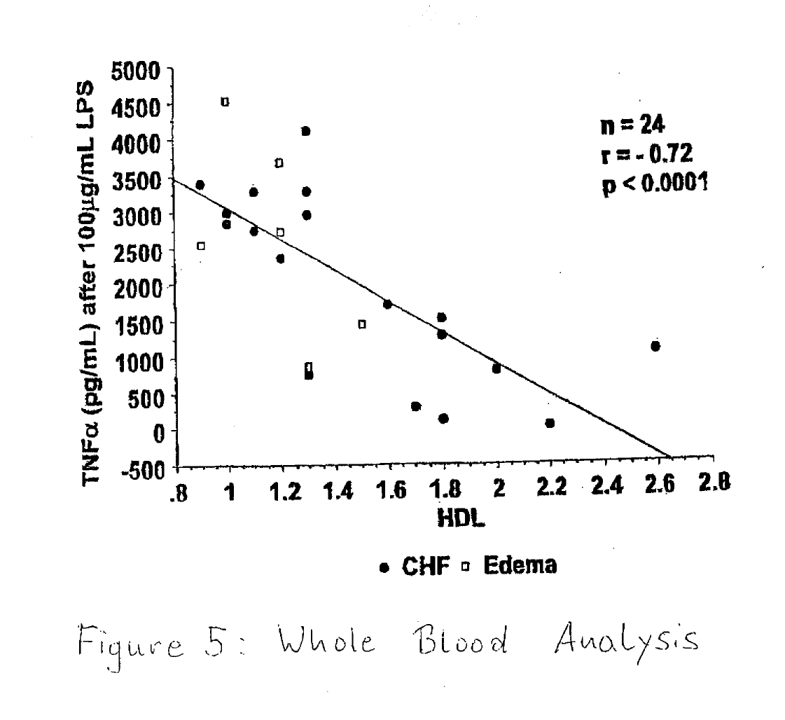

FIG. 5 is a graphical representation showing reduction of TNF-α release upon increased concentration of high-density lipo proteins in a whole blood sample;

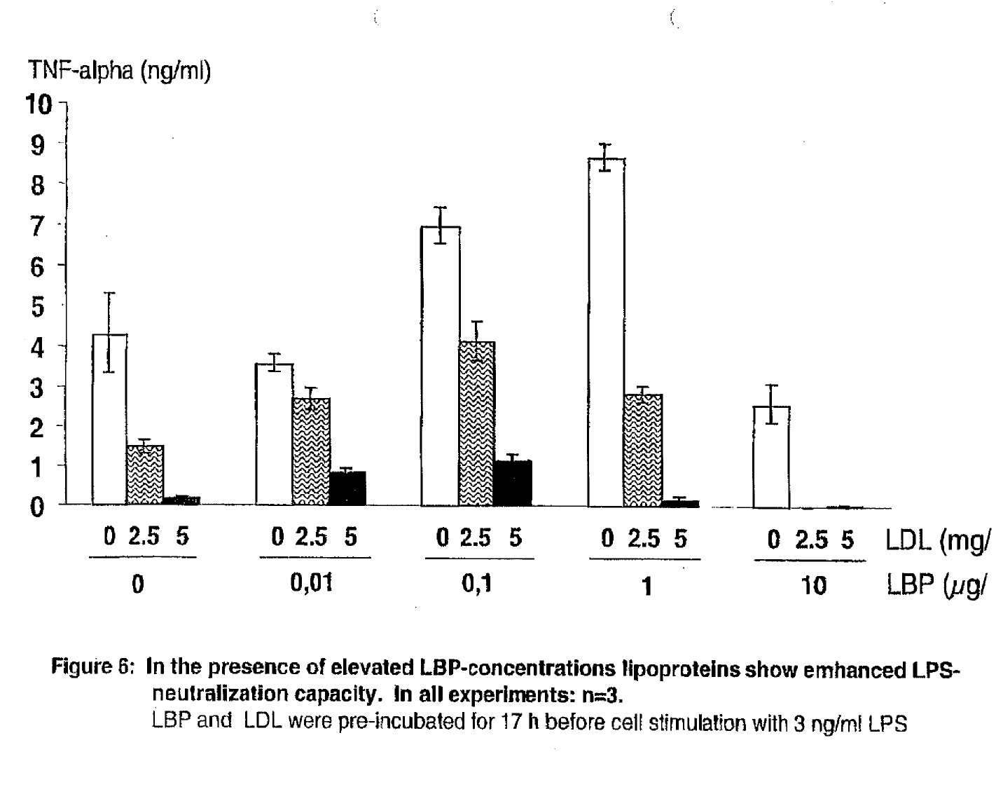

FIG. 6 is a graphical representation showing that low-density lipoproteins show enhanced neutralization capacity in the presence of increased LBP-concentrations;

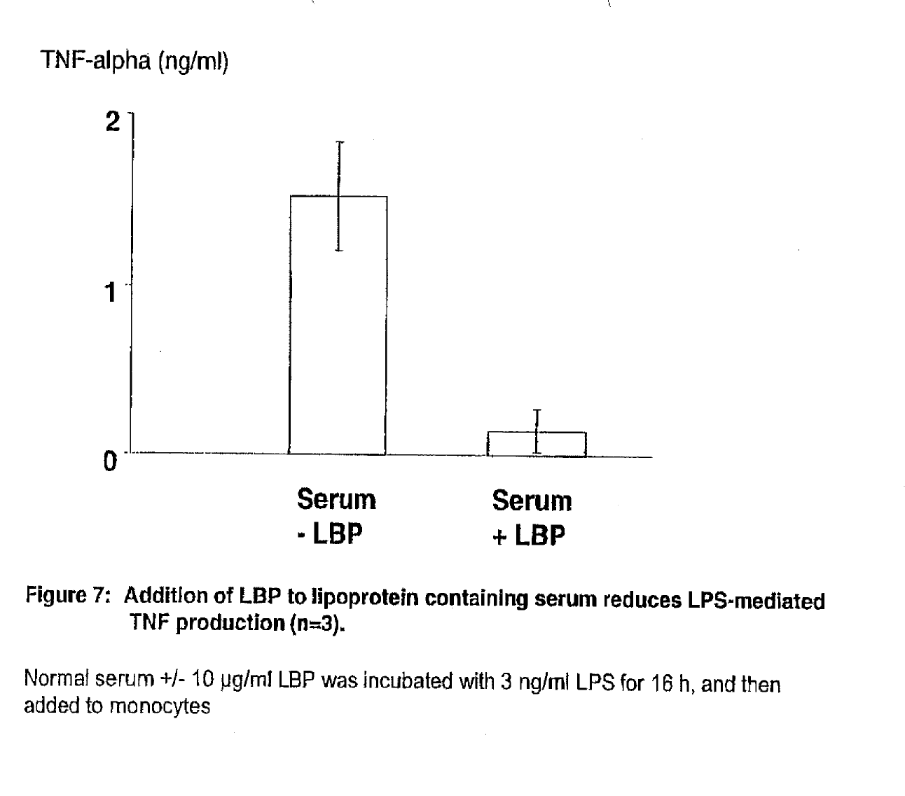

FIG. 7 is a graphical representation showing that addition of LBP to a lipoprotein containing serum reduced LPS-mediated TNF-α production;

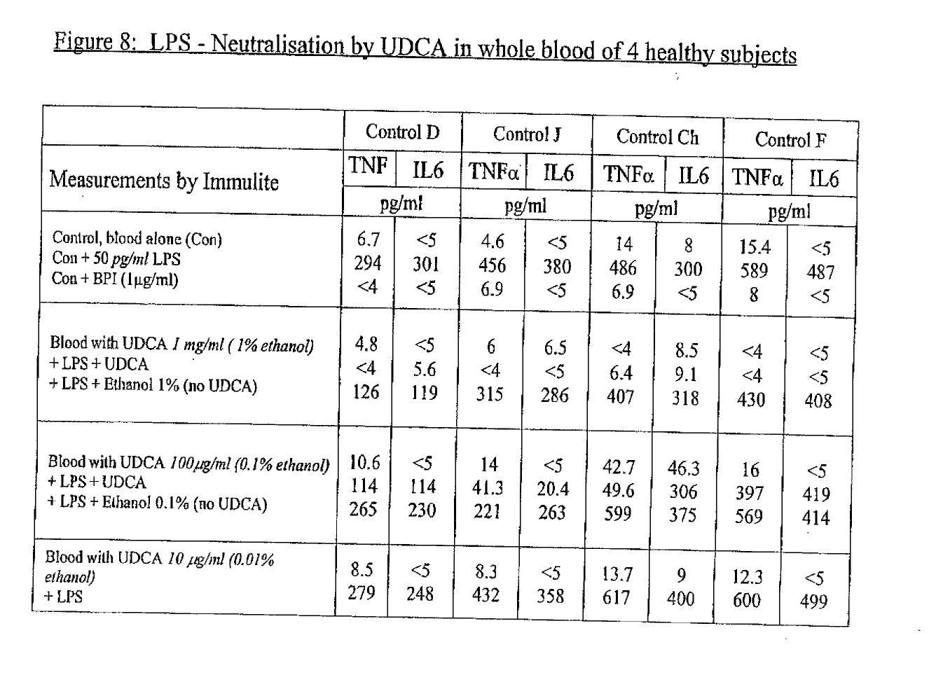

FIG. 8 is a table showing LPS neutralization by UDCA in whole blood of four healthy patients;

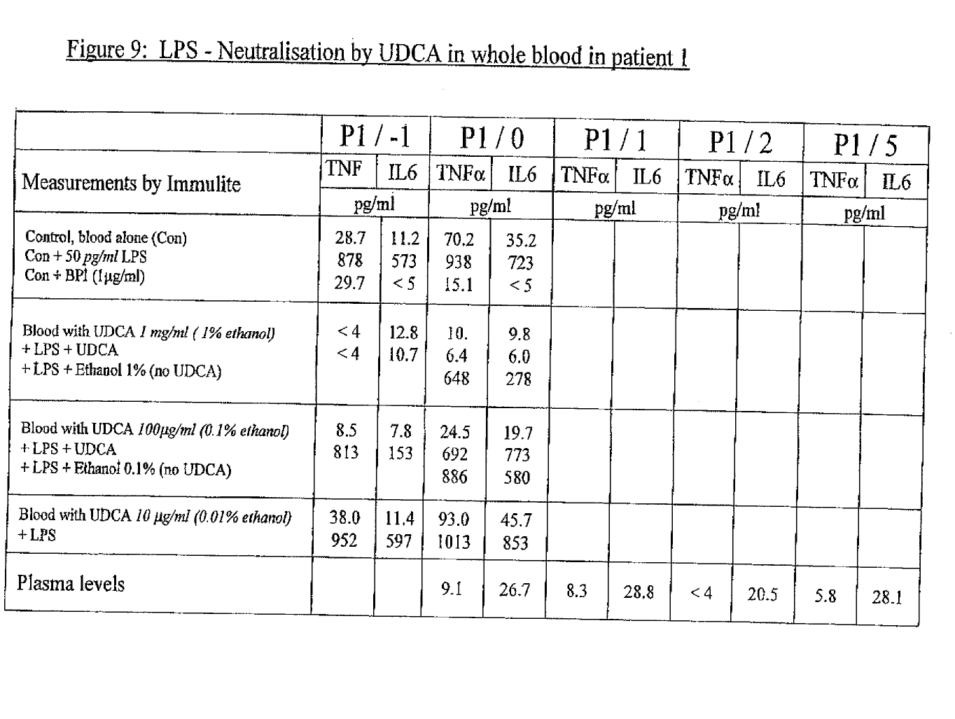

FIG. 9 is a table showing LPS neutralization by UDCA in whole blood of patient 1;

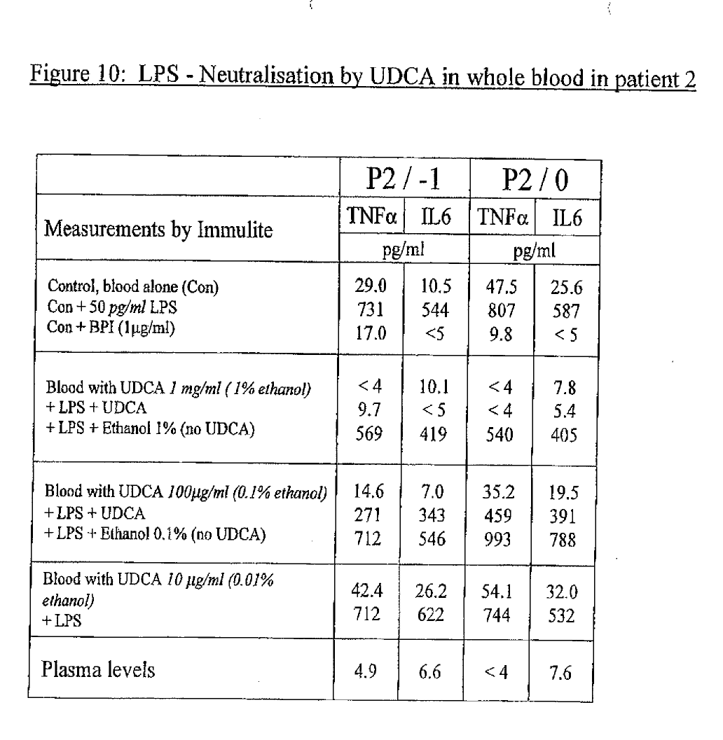

FIG. 10 is a table showing LPS neutralization by UDCA in whole blood of patient 2;

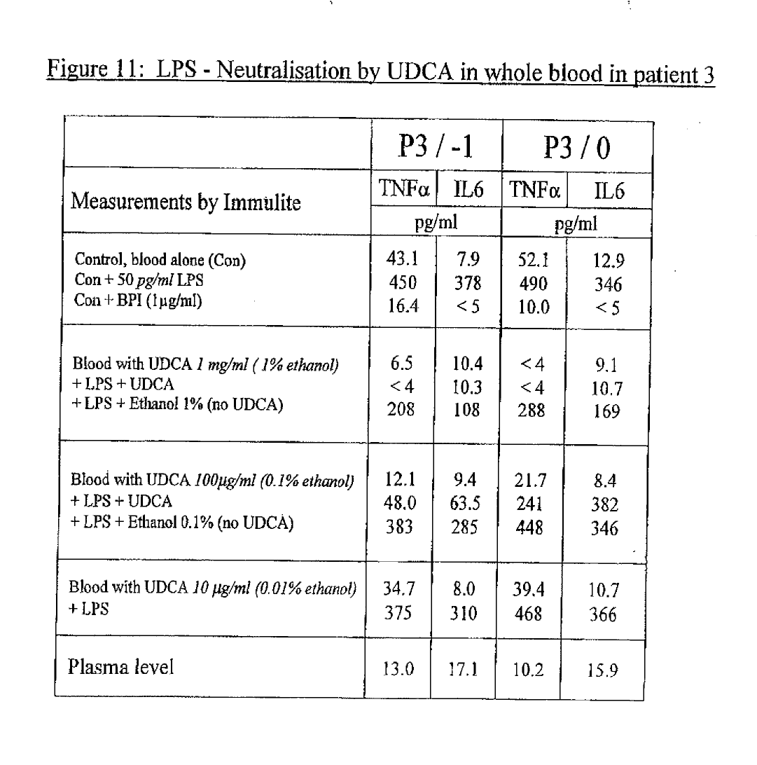

FIG. 11 is a table showing LPS neutralization by UDCA in whole blood of patient 3;

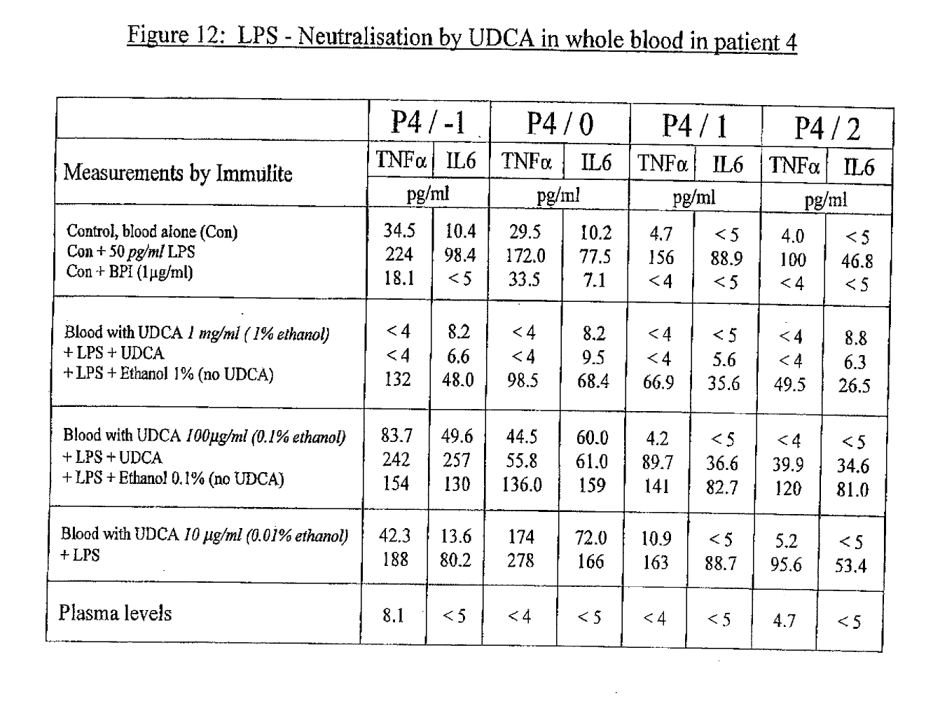

FIG. 12 is a table showing LPS neutralization by UDCA in whole blood of patient 4;

### EXAMPLE 1

We have tested the ability of ursodeoxycholic acid (UDCA, FALK Pharma GmbH) and BPi to inhibit LPS-mediated TNF production in whole blood of patients with cachexia. We studied 4 patients with cachexia due to liver cirrhosis. The patients had all weight loss >7.5% compared to their previous normal weight. In 3 of the 4 patients had a alcoholic aetiology. All patients were studied twice on 2 subsequent days (day “−1” and day “0”), see FIG. 9 to 12.

Methods: Heparinized whole blood was diluted 1:10 with medium ±LPS (50 μg/ml), ±BPi (1 μg/ml), and ±UDCA (1 μg/ml-1 mg/ml) according to the manufacturer's recommendation (Milenia whole blood assay; DPC Biermann, Bad Nauheim, Germany) and incubated for 4 hours at 37° C. In the supernatant, we assessed concentrations of TNF and IL-6 using the semiautomated Immulite system (DPC-Biermann, Bad Nauheim, Germany).

Results: In patients with cachexia due to liver cirrhosis spontaneous (“Control” data) and LPS-stimulated production of TNF and IL6 is significantly elevated compared to that of healthy subjects. LPS-stimulated cytokine production was inhibited by UDCA independently of the effects of the ethanol solution. The detailed results are presented in FIG. 9 to 12. 11 mg/ml UDCA reduced LPS-stimulated TNF production on average by >99% and IL6 production by 97% (ethanol 1% alone on average only by 38% for TNF and 43% for IL6). 100 ug/ml UDCA reduced LPS-stimulated TNF and IL6 production by 42% and 13%, respectively, ethanol 0.1% alone on average only 9% for TNF and IL6 production increased by 18% for ethanol alone). BPi (1 ug/ml) reduced significantly the spontaneous production of TNF and IL6 of whole blood of patients with cachexia due to liver cirrhosis. In 8 experiments 6 times TNF and IL6 levels, respectively, were lowered by at least 5 pg/mi or towards non-detectability, and only in 2 cases TNF and IL6 levels remained stable (p<0.05 for changes).

Conclusion: This is the first documentation that LPS-stimulated cytokine production of whole blood of patients with cachexia can be inhibited by in vitro application of ursodeoxycholic acid (UDCA). This is the first documentation that spontaneous production of inflammatory cytokines in whole blood of patients with cachexia can be inhibited by application of BPi in vitro.

### EXAMPLE 2

We have tested the ability of the therapeutic application of ursodeoxycholic acid (UDCA, FALK Pharma GmbH) to lower plasma levels of TNF and IL6 and to lower spontaneous and LPS-stimulated whole blood cytokine production in patients with cachexia.

We studied in 2 patients with cachexia due to liver cirrhosis plasma cytokine levels after treatment with 3 times 250 mg daily UDCA (FALK Pharma GmbH). The patients had weight loss >7.5% compared to their previous normal weight. The patients were studied at baseline prior to the treatment on 2 subsequent days (day ”−1” and day “0”), and then they were restudied on day 1 (“1”), day 2 (“2”), and day 5 (“5”), see FIGS. 9 and 12.

Methods: Heparinized whole blood was diluted 1:10 with medium ±LPS (50 pg/ml), ±BPI (1 ug/ml), and ±UDCA (1 ug/ml-1 mg/ml) according to the manufacturer's recommendation (Milenia whole blood assay; DPC Biermann, Bad Nauheim, Germany) and incubated for 4 hours at 37° C. In the supernatant and in plasma, we assessed concentrations of TNF and IL-6 using the semiautomated Immulite system (DPC-Biermann, Bad Nauheim, Germany).

Results: Only patient 1 showed elevated plasma levels at baseline (FIG. 9). During 5 days of treatment plasma levels of TNF were lower. In patient 4 we were able to reassess whole blood TNF and IL6 production after 1 and 2 days of treatment with MCA. Spontaneous production of TNF and IL6 in whole blood was reduced substantially to almost undetectable 30 levels. After 2 days of UDCA treatment LPS-stimulated cytokirie production was found to be lowered by 43.5% for TNF and by 39.6% for IL6.

Conclusion: This is the first documentation that US-stimulated cytokine production of whole blood of patients with cachexia can be inhibited by in vivo therapeutic application of ursodeoxycholic acid (UDCA). This is the first documentation that plasma levels of TNF alpha of patients with cachexia can be inhibited by application of BPi.

### EXAMPLE 3

### Endotoxin in Cachectic Patients with Liver Cirrhosis

It has never been studied, whether endotoxin (LPS) or a marker of endotoxaemia may be raised in patients with liver cirrhosis who suffer from cachexia. Plasma levels of soluble CD14 (sCD14) can reflect the history of LPS—cell interaction (Anker et al., Am J Cardiol 1997; 79:1426-1430.).

We investigated in 46 patients with liver cirrhosis (54±12 years, female 15, male 31, Child A:B:C=24:13:9), alcoholic aetiology in 32 patients) resting energy expenditure (REE, indirect calorimetry), food intake diaries, fat mass (skin fold thickness and calculation according to standard formulae) and body cell mass (BCM, body impedance, Data Input 2000, USA).

Soluble CD14 was measured by ELISA (R&D Systems). The majority of patients had a BCM of <35% of body weight (mean standard deviation: 25±7%, median 33%, range 11.8-41.9%).

Plasma sCDI4 levels were significantly increased in patients (mean±standard deviation: 4045±623 pg/ml, median 3920 pg/ml, range 2960-5460 pg/ml) compared to sCD14 levels of healthy individuals (mean: 2714 pg/ml, upper limit of normal 3711 pg/ml, as published in Anker et al., Am. J Cardiol 1997; 79;1426-1430).

The patients with low BCM relative to their body weight must be considered to suffer from wasting disease, which was the majority in this study (63% of patients had a BCM <35%/kg body weight). The majority of patients in this study were metabolically catabolic as evidenced by a REE/BCM coefficient of 67±19 kcal/kg BCM (range 43-163, normal range in healthy subjects: 45-55 kcal/kg).

The strongest correlation that we found was between the degree of wasting (BCM per kg body weight) and the marker of endotoxaemia, i.e. soluble CD14 (r=−0.565, p<0.001). This means, the lower the relative BCM (i.e. the more cachectic) a patient was the higher the were also the sCD14 plasma levels. Plasma levels of sCDI4 also correlated closely and directly with the degree of catabolic energetic/metabolic status (i.e. the REE/BCM coefficient), r=0.549, p<0.001.

Conclusion: This is the first study suggesting that endotoxin (LPS) levels in patients with liver cirrhosis may be particularly high in patients with cachexia. This study also suggests that endotoxin (LPS) is causally related to the characteristics of the cachexia syndrome in liver cirrhosis, i.e. reductions in muscle tissue and increases in metabolic rate.

### EXAMPLE 4

### LBP in Cachectic Patients Due to Liver Cirrhosis

We have studied LSP plasma levels in 6 patients with cachexia due to liver cirrhosis. The patients had weight loss >7.5% compared to their previous normal weight. The disease aetiology was thought to be alcoholic in 4 cases and non-alcoholic in 2 cases. In non of these patients increased LBP levels were found (all below 20 ug/ml). High levels LBP can (together with lipoproteins) block LPS mediated production of inflammatory cytokines. We conclude that LBP is lacking in patients with cachexia due to liver cirrhosis, and that the application of LBP, possibly together with lipoproteins, could counteract the inflammatory to status seen in these patients.

1-24. (canceled)

25. A method for determining, ameliorating, and treating endotoxin-mediated cachexia in a human patient, the method comprising the steps of measuring the level of a cytokine or an inflammatory marker or its production in the blood of the patient and if any such level is elevated, administering to the human patient a therapeutically effective amount of ursodeoxycholic acid.

26. The method according to claim 25, wherein cachexia is due to liver cirrohsis, chronic obstructive pulmonary disease, chronic renal failure, diabetes and rheumatoid arthritis.

27. The method according to claim 25 wherein the ursodeoxycholic acid is able to bind to an endotoxin (lipopolysaccharide) molecule.

28. The method according to claim 25 wherein the ursodeoxycholic acid is able to reduce the available endotoxin in the patient.

29. The method according to claim 25, wherein the level of TNFα, IL-6, CD14, CrP, procalcitonin, lipopolysaccharide levels, and white cell count in the blood of a patient are measured.

30. The method according to claim 25 wherein the ursodeoxycholic acid is administered orally.

31. The method according to claim 25 wherein the ursodeoxycholic acid is administered intravenously.

32. The method according to claim 25 wherein the ursodeoxycholic acid is administered rectally.
